(19) United States
(12) Patent Application Publication
(10) Pub. No.: US 2011/0280765 A1
(43) Pub. Date: Nov. 17, 2011

(54) STERILISER WITH EXHAUST GAS CLEANING SYSTEM FOR DECOMPOSING NOX WITH OZONE

(75) Inventors: Tomoyuki Hirose, Wakayama (JP); Ryuichi Iwasaki, Wakayama (JP); Kazuhiro Kimura, Wakayama (JP); Shigeki Okumura, Wakayama (JP); Kiyotaka Arai, Wakayama (JP)

(73) Assignee: SAIAN CORPORATION, Wakayama-shi, Wakayama (JP)

(21) Appl. No.: 13/145,977

(22) PCT Filed: Mar. 4, 2010
(86) PCT No.: PCT/JP2010/054071

§ 371 Date: Jul. 22, 2011

(30) Foreign Application Priority Data
Mar. 4, 2009 (JP) 2009050679
Sep. 7, 2009 (JP) 2009205705
Mar. 4, 2010 (JP) PCT/JP2010/054071

(51) Int. Cl. A61L 9/04; A61L 9/12

(52) U.S. Cl. 422/30; 422/168

(57) ABSTRACT

An exhaust system is provided for exhausting an exhaust gas used for sterilizing an item to be sterilized by using a high concentration NO₂ gas, including an ozone generator, a gas treatment means for adsorbing ozone generated by the ozone generator and NO₂ in the exhaust gas and accelerating generation of dinitrogen pentoxide or nitric acid by a reaction of the ozone and NO₂ to retain the resultant, and an exhaust apparatus for exhausting the exhaust gas. The exhaust system can effectively and ably purify a high concentration NO₂ exhaust gas having a concentration beyond the normal level.

### TECHNICAL FIELD

The present invention relates to a sterilizing apparatus used for medical instruments such as scissors and catheters for medical use and other items to be sterilized that require increased sterilizing reliability, an exhaust system incorporated into the sterilizing apparatus, and a sterilization method performed by using the sterilizing apparatus.

### BACKGROUND ART

Conventionally, a gas sterilization means for killing such as bacteria and viruses present on an item to be sterilized has been widely used in which an item to be sterilized such as medical instruments is contained in the atmosphere of a sterilant gas such as nitrogen oxide (hereinafter, also simply referred to as “NOx”), ozone, and H₂O₂ for a certain time period. However, since many of sterilization processes are performed in such as hospitals and laboratories in urban areas, a used sterilant gas needs to be released into the atmosphere after a neutralizing process is performed.

For example, in a sterilizing apparatus using a sterilant gas of nitrogen monoxide (hereinafter, also simply referred to as “NO”) or a mixture of NO and nitrogen dioxide (hereinafter, also simply referred to as “NO₂”) which is generated by a sterilizing gas generating composition such as carbon-based diazeniumdiolate compound, a treatment method has been employed by which the sterilant gas is removed by using an oxidant for converting NO into NO₂ and an absorbent for collecting NO₂. By using such method, it is reported that concentrations of NO, NO₂, and NOx can be decreased to the LTWA level (Japanese Unexamined Patent Publication No. 521118/2007).

Alternatively, in a sterilizing apparatus using ethylene oxide gas, an exhaust gas is purified by circulating the exhaust gas multiple times in a gas processing tank which is filled with an adsorbent such as activated charcoal, water, and dilute nitric acid, or a thermal catalytic agent to adsorb ethylene oxide gas (Japanese Unexamined Patent Publication No. 312709/2000).

On the other hand, as a removal method of NOx in an exhaust gas of a diesel engine, a so-called SCR method has been employed in which an SCR catalytic agent is provided in an exhaust passage of an engine, and a reducing agent feeding apparatus for supplying a reducing agent such as ammonia is provided at the upstream side of the SCR catalytic agent to reduce NOx in the exhaust gas with ammonia supplied from the reducing agent feeding apparatus by a catalytic action of the SCR catalytic agent (Japanese Unexamined Patent Publication No. 303826/2000).

### DISCLOSURE OF THE INVENTION

The present inventors invented a high concentration NO₂ generating method which has been filed in another patent application, and has developed a sterilizing apparatus with substantially increased reliability by using a high concentration NO₂ gas obtained by the above method. However, since an exhaust gas exhausted from the sterilizing apparatus contains a high concentration NO₂ gas, the exhaust gas cannot be made to be harmless to a safe level within a predetermined time by any of the conventional methods and apparatus for processing NOx. As a result, there has been a problem that the sterilizing apparatus using the high concentration NO₂ gas cannot be practically used in such as medical sites.

In view of the above situations, on the basis of providing an ozone generator, an exhaust gas treatment means utilizing a converting reaction of dinitrogen pentoxide (N₂O₅) or nitric acid (HNO₃) by means of ozone or NO₂, and an exhaust apparatus for exhausting the exhaust gas, the present invention aims to provide an exhaust system which can efficiently and reliably purify a high concentration NO₂ exhaust gas having a concentration beyond the normal level.

The exhaust system of the present invention is an exhaust system for exhausting an exhaust gas used for sterilizing an item to be sterilized by using a high concentration NO₂ gas, including an ozone generator, a gas treatment means for adsorbing ozone generated by the ozone generator and NO₂ in the exhaust gas and accelerating generation of dinitrogen pentoxide or nitric acid by a reaction of the ozone and NO₂ to retain the resultant, and an exhaust apparatus for exhausting the exhaust gas.

In other words, the present invention is an exhaust system for exhausting an exhaust gas used for sterilizing an item to be sterilized by using a high concentration NO₂ gas, including an ozone generator, a gas treatment means for adsorbing ozone generated by the ozone generator and NO₂ in the exhaust gas and accelerating generation of dinitrogen pentoxide or nitric acid by a reaction of the ozone and NO₂ to retain the resultant, and an exhaust apparatus for exhausting the exhaust gas.

Preferably, the ozone generator includes an ozonizer and an ozone chamber for storing ozone generated by the ozonizer.

Preferably, a buffer portion for adjusting a mixture ratio of NO₂ in the exhaust gas and ozone is further provided at an upstream side from the gas treatment means.

Preferably, the gas treatment means uses an adsorption catalyst.

Preferably, the exhaust apparatus exhausts the exhaust gas used for sterilization in multiple times.

The present invention is a sterilizing apparatus including the following configuration. The sterilizing apparatus includes (a) an NO₂ gas supply system configured by a circulating path in which a chamber for storing a high concentration NO₂ gas, a plasma generator, and a circulating means are connected, (b) a sterilizing chamber connected to the chamber via a first open/closure device, and (c) the exhaust system connected to the sterilizing chamber via a second open/closure device.

Preferably, the chamber is connected to the exhaust system via a third open/closure device.

Preferably, the sterilizing chamber is provided with a measuring path for returning the high concentration NO₂ gas in the sterilizing chamber to the sterilizing chamber through an NO₂ sensor.

Preferably, a plurality of sterilizing chambers is connected to a single exhaust system.

In addition, the present invention is a sterilizing method using the sterilizing apparatus, wherein in a gas exhausting step for exhausting a high concentration NO₂ gas used for sterilizing an item to be sterilized, the high concentration NO₂ gas is exhausted to the exhaust system in part with a predetermined NO₂ gas contents.

A sterilizing method using the sterilizing apparatus, wherein in a gas exhausting step for exhausting a used high concentration NO₂ gas after a sterilizing step for sterilizing an item to be sterilized, a procedure of exhausting the high concentration NO₂ gas in the sterilizing chamber to the exhaust gas treatment means by an exhaust apparatus of the exhaust system to obtain a negative pressure in the sterilizing chamber while the first open/closure device is closed and the second open/closure device is open, and subsequently, sucking the high concentration NO₂ gas remaining the chamber into the sterilizing chamber by the negative pressure by opening the first open/closure device and closing the second open/closure device is repeated.

In addition, the present invention is a sterilizing method using the sterilizing apparatus, including the steps of (d) setting an item to be sterilized in the sterilizing chamber (setting step), (e) vacuuming the inside of the sterilizing chamber (vacuuming step), (f) humidifying the inside of the sterilizing chamber (humidifying step), (g) opening the first open/closure device to supply the NO₂ gas generated by the NO₂ gas system and stored in the chamber to the sterilizing chamber (supplying step), (h) filling the dried gas mixture in the chamber (air charging step), and (i) generating NO₂ gas by driving the NO₂ gas supply system (circulating step), wherein the steps (g) to (i) are repeated a plurality of times.

Preferably, before the air charging step of step (h), a step of closing the first open/closure device and opening the third open/closure device to directly couple the chamber and exhaust system to exhaust the NO₂ gas remaining in the chamber with the exhaust system and to vacuum the chamber (gas exhausting step) is performed.

### BRIEF DESCRIPTION OF THE DRAWINGS

FIG. 1 is an explanatory view illustrating a configuration of the exhaust system according to one Embodiment of the present invention.

FIG. 2 is an explanatory view illustrating a plasma generating portion in a configuration of the exhaust system according to one Embodiment of the present invention.

FIG. 3 is an explanatory view illustrating a plasma generator in a configuration of the exhaust system according to one Embodiment of the present invention.

FIG. 4 is an explanatory view illustrating an exhaust system according to one Embodiment of the present invention.

### BEST MODE FOR CARRYING OUT THE INVENTION

Hereinafter, Embodiments of the present invention are described by referring to the drawings. The exhaust system is a system by which an exhaust gas used for sterilizing an item to be sterilized with a high concentration NO₂ gas is exhausted. In the present Embodiment, the system is employed in a sterilizing apparatus which is capable of efficiently perform sterilizing process on medical instruments and other items to be sterilized. Hereinafter, an Embodiment of the sterilizing apparatus is described first.

In the sterilizing apparatus of the present Embodiment, a sterilizing apparatus is illustrated, the apparatus being configured to include an NO₂ gas supply system for generating a high concentration NO₂ gas, a sterilizing chamber for sterilizing an item contained to be sterilized with the high concentration NO₂ gas, and an exhaust system for making an exhaust gas which is the high concentration NO₂ gas used for sterilization to be harmless.

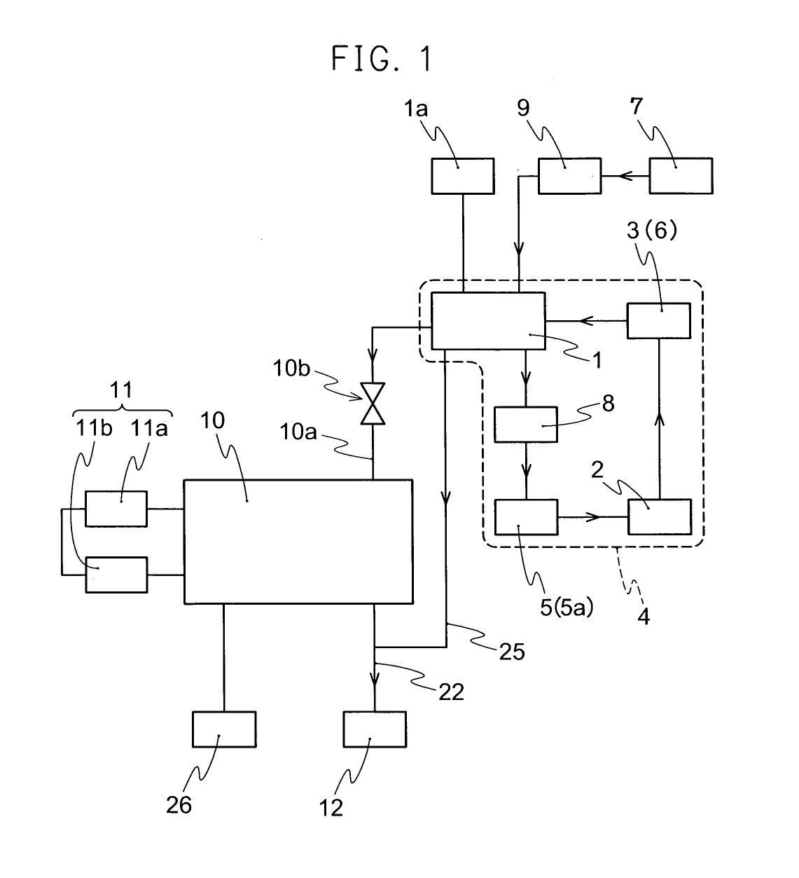

As shown in FIG. 1, the NO₂ gas supply system includes a circulating path 4 connecting a chamber 1, a plasma generator 2, and a circulating means 3. More specifically, the circulating path 4 is configured to include the chamber 1, a flow resistive portion 5 connected to the chamber 1 at the downstream side of the path via a pipe, the plasma generator 2 connected to the flow resistive portion 5 at the downstream side of the path via a pipe, and the circulating means 3 connected to the plasma generator 2 at the downstream side of the path via a pipe. The circulating means 3 is further connected to the chamber 1 at the upstream side of the path via a pipe such that a cyclic circulating path 4 is formed by the chamber 1, flow resistive portion 5, plasma generator 2, and circulating means 3. By the operation of the circulating means 3, a gas mixture including nitrogen and oxygen circulates in the circulating path 4 to generate NO₂.

In the present description, a gas including nitrogen and oxygen which is supplied from the outside to the high concentration NO₂ generating system as an ingredient is referred to as a gas mixture, a gas including NOx which is generated by circulating through the plasma generator 2 at least once is referred to as an NOx gas mixture, and a gas which reached a desired level of NO₂ concentration by repeating the above-described circulation is referred to as a high concentration NO₂ gas.

The chamber 1 is an airtight compartment for containing high concentration NO₂ gas to be generated. The chamber 1 has a rectangular box shape in the present Embodiment, however, it may have a spherical shape or cylindrical shape. Since the chamber 1 of the present Embodiment forms the circulating path 4, a flow outlet, a flow inlet, and an openable and closable gas supply opening for taking out high concentration NO₂ are formed.

In the present Embodiment, the flow resistive portion 5 is formed by an orifice 5a. The orifice 5a is provided in the pipe at the downstream side from the chamber 1, and makes up an orifice fluid meter. In the present Embodiment, therefore, it is advantageous that the flow volume of the gas circulating out of the chamber 1 can be measured. In addition to the orifice 5a, the flow resistive portion 5 may be configured in such a manner that a portion of the pipe at the downstream side from the chamber 1 is configured by a narrow pipe to increase the flow resistivity of that portion.

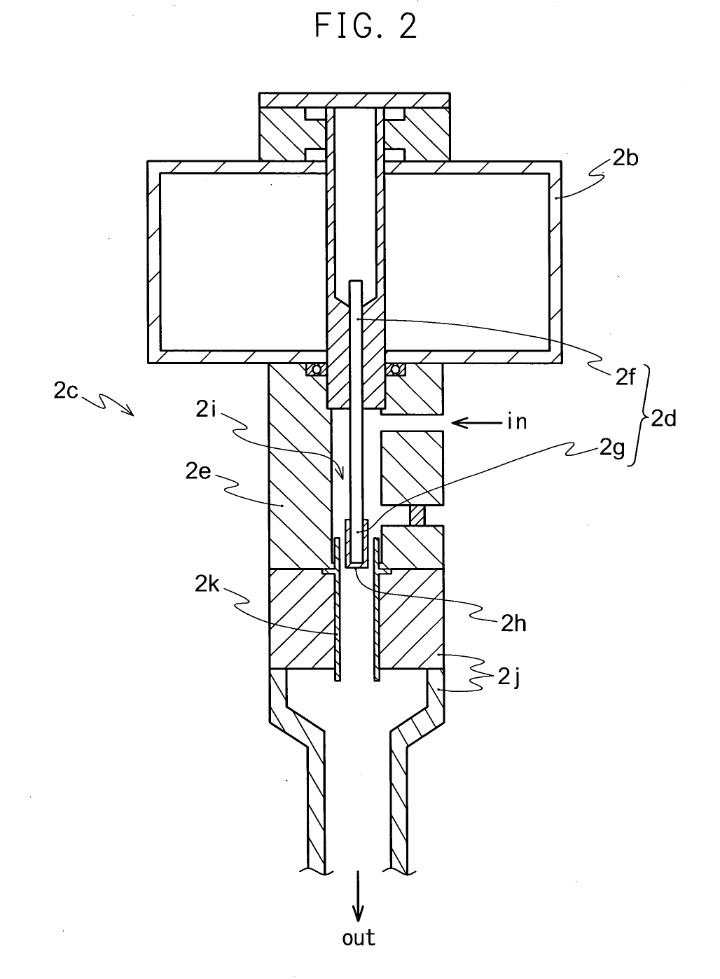

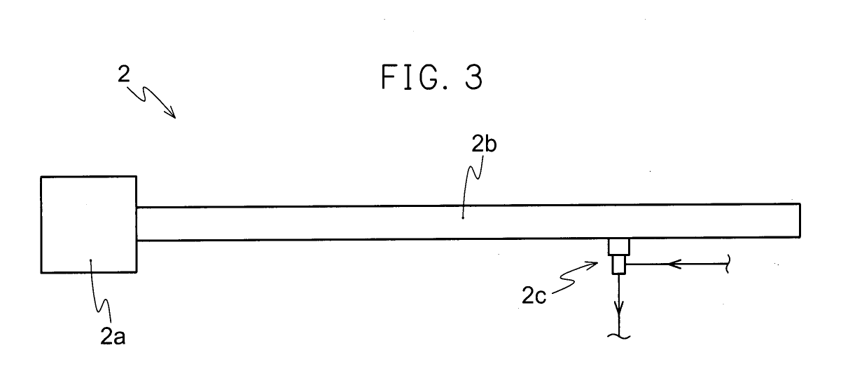

As shown in FIGS. 2 and 3, the plasma generator 2 is a unit capable of generating a plasma under normal temperature and normal pressure by using microwaves, and is generally configured to include a microwave generating apparatus 2a for generating microwaves with a predetermined wavelength, a waveguide 2b which is connected to the microwave generating apparatus 2a to transmit the microwaves, and a plasma generating portion 2c which is provided integrally with the waveguide 2b.

The microwave generating apparatus 2a generates microwaves at 2.45 GHz, for example, and transmits the microwaves into the waveguide 2b. The microwave generating apparatus 2a, therefore, includes a microwave generating source such as a magnetron for generating microwaves, an amplifier for adjusting the power of the microwaves generated at the microwave generating source to a predetermined power intensity, and a microwave transmitting antenna for emitting the microwaves into the waveguide 2b. As the microwave generating apparatus 2a used in the plasma generator 2, an apparatus of a continuous variable type which is capable of outputting microwave energy between of 1 W and 3 kW, for example, is suitable.

The waveguide 2b is made of a nonmagnetic metal (such as aluminum), for example, has a tubular shape with a rectangular cross section, and transmits the microwaves generated by the microwave generating apparatus 2a toward the plasma generating portion 2c. The waveguide 2b of the present Embodiment is configured by a square tubular assembly using top and bottom plates, and two side plates made of metallic flat plates. In addition to such plate assembly, the waveguide may also be formed by such as extrusion or bending process of a plate member. Moreover, a waveguide 2b with an oval cross section may be used in addition to the waveguide 2b with a rectangular cross section. Furthermore, not only by nonmagnetic metals, but the waveguide may be configured by various members having the waveguide property. The waveguide 2b is grounded in the present Embodiment.

The plasma generating portion 2c is integrally configured with the waveguide 2b, and includes a rod-shaped conducting shaft 2d inserted through the waveguide 2b and a tubular conducting tube 2e. The conducting shaft 2d is further configured by an antenna portion 2f which is inserted into the waveguide 2b to receive the microwaves, and a center electrode 2g protruding externally from the waveguide 2b which, in the present Embodiment, is inserted through the waveguide 2b via an electric insulator. The conducting shaft 2d of the present Embodiment has a circular cross section, however, the conducting shaft with an elliptical, oval, or a long oval cross section may be employed. The conducting shaft 2d of the present Embodiment is formed by using titanium, however, materials capable of conducting microwaves such as titanium alloy, copper, platinum, gold, and silver may be used. A shielding film 2h made of ceramic is formed at a tip of the conducting shaft 2d (center electrode 2g) for preventing arc discharge and protecting the electrode.

In the present Embodiment, the conducting tube 2e has a generally cylindrical shape, and the inner diameter thereof is formed to be larger than the outer diameter of the conducting shaft 2d. The conducting tube is provided to cover the center electrode 2g protruding externally from the waveguide 2b while having the center electrode at the center, and a ring-shaped space 2i is formed between the center electrode 2g and conducting tube 2e. The base end of the conducting tube 2e is electrically conductive and fixed relative to the waveguide 2b, and the conducting tube 2e is thus grounded via waveguide 2b. The conducting tube 2e may have such as a rectangular cross section or an oval cross section in addition to a circular cross section. However, a tip thereof is formed to have a length which terminates with substantially the same position as a tip of the center electrode 2g. It is noted that the conducting tube 2e of the present Embodiment is made using stainless steel, however, it may be made of such as aluminum.

In the conducting tube 2e of the present Embodiment, a ventilation opening is provided at a position toward the base end thereof. By connecting a pipe 2j extending from the flow resistive portion 5 to the ventilation opening, the circulating path 4 connecting from the flow resistive portion 5 to the plasma generator 2 is configured. The gas mixture flowing in the circulating path 4 moves through inside the ring-shaped space 2i toward the end of the center electrode 2g. Furthermore, to an outside edge of the conducting tube 2e, a ceramic shielding tube 2k is inserted to prevent the arc discharge relative to the center electrode 2g. The outside edge of the shielding tube 2k is connected to the pipe 2j directing further toward the downstream of the path to thereby form the circulating path 4.

In the plasma generating portion 2c thus configured, 2.45 GHz of microwave (power is adjustable) generated from the microwave generating apparatus 2a (magnetron) is emitted from the microwave transmitting antenna of the microwave generating apparatus 2a provided at one end of the waveguide 2b to the plasma generating portion 2c. The emitted microwave transmits in the waveguide 2b and is received by the antenna portion 2f of the conducting shaft 2d in the plasma generating portion 2c. The microwave thus received by the antenna portion 2f transmits on the surface of the conducting shaft 2d, and reaches the tip of the center electrode 2g. The tip of the center electrode 2g is electrically coupled to the waveguide 2b, and is disposed nearby the tip of the conducting tube 2e of a ground potential. By the microwaves reached the tip of the center electrode 2g, an intense electric field is formed between the tip of the conducting tube 2e and the tip of the center electrode 2g, especially in the vicinity of the tip of the center electrode 2g. It is noted that the conducting shaft 2d is formed to have a resonance point in the 2.45 GHz band, such that an intense electric field is efficiently formed at the tip portion of the center electrode 2g.

By the intense electric field thus formed, partial ionization is generated in nitrogen and oxygen included in the gas mixture supplied via the circulating path 4. As a result, an aggregate of electrons at several tens of thousands degrees in Celsius, ions at substantially normal temperature, neutral atoms at substantially normal temperature, and neutral molecules at substantially normal temperature is composed. Comprehensively, this condition is electrically neutral, and in other words, a plasma state, and more particularly a low-temperature plasma (non-equilibrium plasma) state is formed.

In other words, nitrogen and oxygen of the gas mixture in the vicinity of the end of the center electrode 2g generate dielectric breakdown by being excited through the intense electric field formed by the microwaves, and are displaced from the molecular state to the low-temperature plasma (non-equilibrium plasma) state. The gas under the low-temperature plasma state has a high reactivity with respect to other gases under the low-temperature plasma state or molecular state. Therefore, when the gas mixture including primarily nitrogen and oxygen is introduced to the plasma generating portion 2c, a portion thereof is converted to nitrogen oxides of such as nitrogen monoxide and nitrogen dioxide or to ozone.

N₂+O₂→2NO 1

N₂+2O₂→2NO₂ 2

3O₂→2O₃ 3

It is noted that the conversion ratio is the largest in the case of equation 1. A portion of nitrogen monoxide generated according to equation 1 binds with oxygen under the low-temperature plasma state in the plasma generating portion 2c and is converted to nitrogen dioxide.

2NO+O₂→2NO₂ 4

The NOx gas mixture including NO₂ thus generated circulates through the circulating path 4 or is retained in the chamber 1. During this time, nitrogen monoxide generated according to equation 1 reacts stepwise with oxygen in the NOx gas mixture or with the ozone generated according to equation 3, and is further converted to nitrogen dioxide as shown in equations 5 and 6. As a result, the NO₂ concentration increases.

2NO+O₂→2NO₂ 5

NO+O₃→NO₂+O₂ 6

Ozone generated according to equation 3 reacts with nitrogen in the NOx gas mixture to generate nitrogen monoxide.

N₂+2O₃→2NO+2O₂ 7

This nitrogen monoxide is also converted to nitrogen dioxide by the reactions according to equations 5 and 6.

In this manner, when the NOx gas mixture repeats to circulate in the circulating path, the concentration of nitrogen dioxide gradually increases and the high concentration NO₂ gas with a desired level of NO₂ concentration is obtained. However, when the generated nitrogen monoxide or nitrogen dioxide again passes through the plasma generator 2, a phenomenon occurs that a portion thereof again becomes under the low-temperature plasma state by a dissociation reaction and thus returns to nitrogen or oxygen. Accordingly, when the concentration of the NOx gas mixture reaches a certain level of the high concentration NO₂ gas by repeating the circulation, the generation of nitrogen oxide and the dissociation of nitrogen oxide fall under an equilibrium state, so that the enhancement does not proceed further at a certain concentration.

In the high concentration NO₂ gas generating system of the present Embodiment, a circulating path 4 including a single plasma generator 2 is illustrated as shown in FIG. 1. However, two or three or more plasma generators 2 may be connected in parallel to form the circulating path 4. This is preferable since the high concentration NO₂ gas can be generated in a short time in such case. Furthermore, the circulating path 4 may be divaricated in the plasma generator 2 to provide a plasma generating portion 2c for each of the diverged paths.

The circulating means 3 is configured by using a pressure device 6 in the present Embodiment. A fan may also be used as the circulating means 3. As the pressure device 6, an air pump may be preferably employed, and an air blower or air compressor may also be used. As for the air pump, a diaphragm pump with approximately 20 to 100 watt power and made of fluorine rubber, a plunger pump made of ceramic, or bellows pump may be employed. The pressure device 6 is provided in the pipe for connecting the plasma generator 2 and the chamber 1, and is connected to apply pressure to the chamber 1 side at the downstream side of the path.

As mentioned above, the high concentration NO₂ gas generating system of the present Embodiment makes up the cyclic circulating path 4 by connecting the chamber 1, flow resistive portion 5, plasma generator 2, and pressure device 6, in circular via the pipes. By the operation of the pressure device 6, the air (gas mixture) introduced from the inlet portion 7 flows through the circulating path 4, and the NOx gas mixture is generated which includes nitrogen monoxide and nitrogen dioxide generated by the reaction of nitrogen and oxygen displaced to the low-temperature plasma (non-equilibrium plasma) state when passing through the plasma generator 2. The nitrogen monoxide is converted to nitrogen dioxide when it reacts with oxygen in the NOx gas mixture and ozone stepwise. The high concentration NO₂ gas can thus be generated by the gradual increase in the concentration of the nitrogen dioxide.

In the present Embodiment, an NO₂ concentration measurement sensor 8 is provided at the downstream of the chamber 1. With this, the concentration of NO₂ can be measured. In addition, a gas drying means 9 for adjusting a dew point of the air to be introduced in the chamber 1. As for the gas drying means 9, a self-regenerating system by means of molecular sieve filled in two tubes may be used, for example. Furthermore, the chamber 1 is provided with pressure meter 1a and plasma generator 2 is provided with pressure meter (not shown), respectively. With those, pressures in the chamber 1 and plasma generator 2 can be controlled, and it is possible to check if the pressure is abnormal.

In the present Embodiment, the sterilizing chamber 10 constitutes a main portion of the sterilizing apparatus for medical instruments, and includes an opening for loading and unloading an item to be sterilized, a shielding door capable of sealing the opening, a gas supply opening for introducing a high concentration NO₂ gas, and a gas exhaust opening for exhausting an exhaust gas after sterilization. The shielding door is provided with a sealing material in the periphery for securing the sealing property. For the sealing material of the present Embodiment, fluorine-containing elastomer is used from the perspective of the pressure tightness and corrosion resistance. It is preferable that the safety is improved when the shielding door is provided with an interlock which does not allow opening the door in the case an NO₂ gas concentration in the sterilizing chamber 10 is equal to or more than the level harmful for humans.

A supply pipe 10a is provided from the gas supply opening to the chamber 1 of the NO₂ gas supply system. The high concentration NO₂ gas stored in the chamber 1 passes through the supply pipe and is supplied from the gas supply opening to the sterilizing chamber 10. Furthermore, the supply pipe is provided with a first open/closure device 10b using an air drive valve, and the supply of the high concentration NO₂ gas to the sterilizing chamber 10 is controlled by an on/off control of the first open/closure device 10b.

The sterilizing chamber 10 is provided with a measuring path which sucks the NO₂ gas in the chamber by a pump to measure the concentration thereof with an NO₂ sensor 11 and returns the gas to the sterilizing chamber 10. In the measuring path of the present Embodiment, the path is diverged into two paths in the middle to respectively provide two NO₂ sensors 11a, 11b for high concentration measurement and low concentration measurement such that measurements of the concentration can be performed with high accuracy.

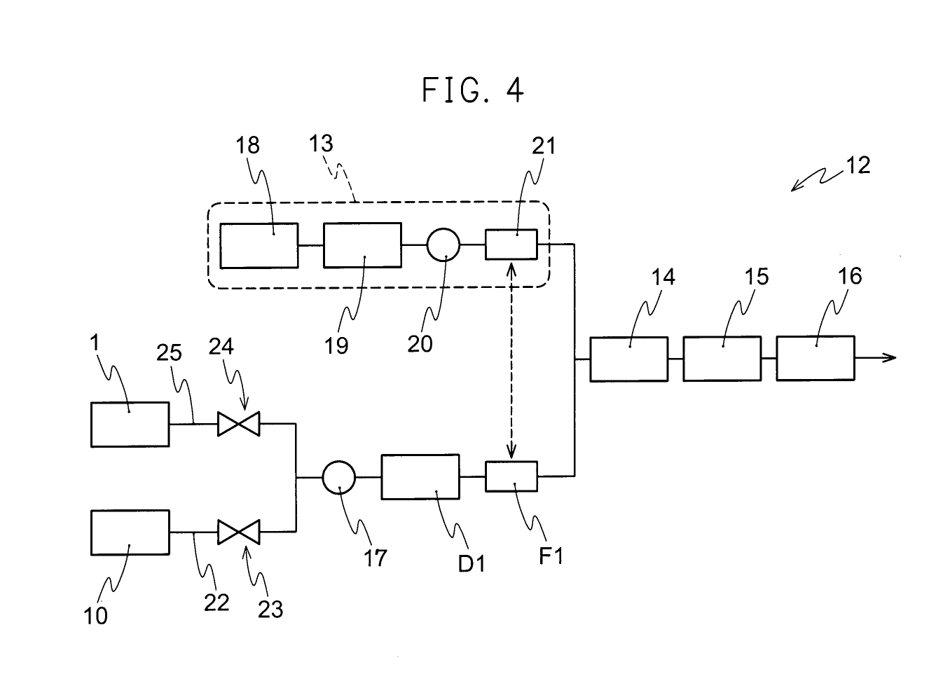

As shown in FIGS. 1 and 4, the exhaust system 12 makes the high concentration NO₂ gas after a sterilization process (exhaust gas) and filled in the sterilizing chamber 10 to be harmless for exhaustion. The exhaust system is configured to include an ozone generator 13, a buffer portion 14 for adjusting a mixture ratio of ozone supplied from the ozone generator 13 and the exhaust gas, a gas treatment means 15, an ozone treatment means 16, and an exhaust apparatus 17. In the downstream from the exhaust apparatus 17 and the upstream of the buffer portion, it is configured to include a dehumidifying portion D1 and an exhaust gas flow meter F1.

The dehumidifying portion D1 is composed of silica gel in the present Embodiment. Silica gel serves to prevent a failure of the exhaust gas flow meter due to the dew drop inside the meter and promote efficiency of the gas treatment means 15 in the downstream by adsorbing the moisture included in the high concentration NO₂ gas (exhaust gas). It is noted that a portion of NO₂ is absorbed by the silica gel. The exhaust gas flow meter F1 measures the flow volume of the exhaust gas to be exhausted.

In the present Embodiment, the ozone generator 13 includes an ozonizer 18, an ozone chamber 19, an ozone exhaust apparatus 20, and an ozone flow controller 21. The ozonizer 18 (ozone generator) is an apparatus for applying a high voltage between electrodes provided with dielectric to discharge the air or oxygen filled in a discharge gap to convert the air or oxygen to ozone. Ozonizers are widely used as environmental equipments.

The ozone chamber 19 is a small space having a container form with approximately 40 to 80 L volume, and is connected to the ozonizer 18 to temporarily store ozone generated by the ozonizer 18. By providing such ozone chamber 19, dinitrogen pentoxide generated by a chemical reaction with the high concentration NO₂ gas or ozone required for nitrification can be supplied by a low-powered ozone generator 13 such that the high concentration NO₂ gas can be securely made to be harmless.

The exhaust volume of ozone from the ozone chamber 19 is adjusted by the ozone exhaust apparatus 20 and the ozone flow controller 21. Specifically, the ozone flow controller 21 is a regulating valve which can control the flow volume of ozone based on the flow volume of the exhaust gas measured by the exhaust gas flow meter F1. Thus, the flow volume of the high concentration NO₂ and the mixture ratio of the ozone flow can be adjusted, and sufficient ozone relative to NO₂ included in the exhaust gas can be delivered.

The buffer portion 14 is a small space having a container form with approximately 10 L volume. Depending on pressure fluctuations in the pump for pressure filling ozone, an amount of ozone being supplied fluctuates to fall in either an excessive or a short side relative to a time axis. However, the amount of ozone being supplied with large fluctuations can be averaged by controlling the flow volume of ozone from the ozone chamber 19 with the ozone flow controller 21 on the basis of the exhaust gas flow meter F1 and feeding the ozone to the buffer portion 14 for mixing, and by retaining the gas mixture in the buffer portion 14.

In the present Embodiment, the gas treatment means 15 is configured by providing a processing portion containing an adsorption catalyst on the exhaust gas path located at the downstream side from the buffer portion 14. The adsorption catalyst is a catalyst for excellently adsorbing NO₂ and ozone, and accelerating the reaction of the adsorbed NO₂ and ozone to chemically convert to N₂O₅, or generating HNO₃ by a reaction of NO₂ with remaining vapors. In the present Embodiment, zeolite is used as an adsorption catalyst. Of zeolite, silicalite is preferably used since NO₂ can be efficiently adsorbed.

The ozone treatment means 16 is located at the downstream from the gas treatment means 15 and serves as an ozone decomposition apparatus for decomposing the excess ozone in the reaction with NO₂. With this arrangement, ozone can be exhausted by controlling its concentration to be a predetermined level or below.

The gas treatment means 15 thus configured adsorbs the gas mixture in which NO₂ and ozone are adjusted to be at a suitable mixture ratio in the buffer portion 14, and chemically converts the gas to N₂O₅ by NO₂ and ozone, or accelerates the generation of HNO₃ by the reaction of NO₂ and remaining vapors. NO₂ in the exhaust gas is thus removed, and the exhaust gas is made to be harmless. In addition, since NO₂ is eliminated by generating N₂O₅ or HNO₃ by the chemical reaction with ozone, NO₂ can be efficiently eliminated even if the concentration of NO₂ is high. In sterilizing apparatus using high concentration NO₂, reliable exhaust gas treatment can be performed within practically suitable time period. Furthermore, in the present Embodiment, an NO₂ sensor and ozone sensor are provided at the downstream side from the ozone treatment means 16 to check whether the exhaust gas has been made to be harmless.

In the gas treatment means 15, since NO₂ and ozone of the gas mixture is chemically converted to generate N₂O₅ or HNO₃, a suitable mixture ratio of NO₂ and ozone in the gas mixture present in the buffer portion 14 is considered to be 2:1. In practical, however, since ozone is likely to decompose under a high pressure environment, and particularly since pipes and each of containers are made of metals, those metals act as a catalyst and the decomposition is further accelerated. The mixture ratio of NO₂ and ozone in the gas mixture is thus made to be such that the ratio of ozone is larger than 2:1, and the mixture ratio is preferably 2:1 to 1:2, for example. In the present Embodiment, the ratio is 3:2.

The exhaust system thus configured is connected to the gas exhaust opening of the sterilizing chamber 10 by an exhaust pipe 22 therebetween. More specifically, the exhaust pipe 22 extending from the sterilizing chamber 10 is connected to the buffer portion 14 such that the exhaust gas exhausted from the sterilizing chamber 10 is transferred to the buffer portion 14. Furthermore, the exhaust pipe 22 is provided with a second open/closure device 23 using an air drive valve, and the transfer of the exhaust gas to the buffer portion 14 is controlled by the on/off control of the second open/closure device 23.

The exhaust apparatus 17 serves to suck the exhaust gas remaining in the sterilizing chamber 10 and transfer the gas to the exhaust system, and after making the gas to be harmless by the gas treatment means 15, imparts energy to the flow of the exhaust gas by using such as an air pump and a fan. In the present Embodiment, an air pump is provided in the exhaust pipe 22.

The exhaust apparatus 17 of the present Embodiment is controlled such that the exhaust gas in the sterilizing chamber 10 is exhausted at once. However, the apparatus may be controlled such that the gas is exhausted to the buffer portion 14 in multiple times. In this manner, in the case the exhaust gas is supplied to the exhaust gas treatment means 15 in multiple times, it is advantageous that the exhaust gas can be reliably made to be harmless.

In the present Embodiment, the chamber 1 of the NO₂ gas supply system and the exhaust system is connected by a bypass pipe 25 provided with a third open/closure device 24. By opening the third open/closure device 24, an exhausting step in which the inside of the chamber 1 is vacuumed can be performed by driving the exhaust apparatus 17 of the exhaust system. In addition, NO₂ gas remaining in the chamber 1 is made to be harmless by the exhaust system to safely exhaust.

In the present Embodiment, the sterilizing apparatus is configured in which one sterilizing chamber 10 and exhaust system are linked via the exhaust pipe 22. Alternatively, a multiple type apparatus in which exhaust pipes 22 from a plurality of sterilizing chambers 10 are linked to a single exhaust system may be provided, and the exhaust system which is only required at the time of exhausting the exhaust gas may be controlled such that the system is driven while being shared. Since a shared exhaust system can serve relative to multiple sterilizing chambers 10, there is no unnecessary part as a whole sterilizing apparatus and the apparatus can be made to be compact.

Hereinafter, an Embodiment of the sterilization method of the present invention by using the sterilizing apparatus is described. The sterilization method including the following steps:

(1) setting an item to be sterilized in the sterilizing chamber 10 (setting step),

(2) vacuuming the inside of the sterilizing chamber 10 (vacuuming step),

(3) humidifying the inside of the sterilizing chamber 10 (humidifying step),

(4) opening the first open/closure device 10b to supply the NO₂ gas generated by the NO₂ gas system and stored in the chamber 1 to the sterilizing chamber 10 (supplying step),

(5) closing the first open/closure device 10b and opening the third open/closure device 24 to directly couple the chamber 1 and exhaust system to exhaust the NO₂ gas remaining in the chamber 1 with the exhaust system and to vacuum the chamber (exhausting step),

(6) filling the dried gas mixture in the chamber 1 (air charging step),

(7) generating NO₂ gas by driving the NO₂ gas supply system (circulating step),

(8) sterilizing the item to be sterilized by the high concentration NO₂ gas filled in the sterilizing chamber 10 (sterilizing step),

(9) exhausting the used high concentration NO₂ gas from the sterilizing chamber 10 (gas exhausting step).

In the setting step, the shielding door of the sterilizing chamber 10 is opened, and the item to be sterilized is placed by inserting it from the opening to the inside. In order not to prevent the contact with the high concentration NO₂ gas, the item to be sterilized may be suitably placed on a placement table in accordance with its shape. In the case a plurality of items to be sterilized is sterilized at the same time, shelves may be arranged in such a manner that they do not overlap each other, and the items are placed thereon.

In the vacuuming step, the pressure of the inside of the sterilizing chamber 10 is decreased through exhausting the air in the chamber by driving an air pump of the exhaust apparatus. Through this depressurization, the air in detailed and innermost portions such as a hole of the item to be sterilized is discharged. When the high concentration NO₂ gas is filled in the subsequent sterilizing step, the NO₂ gas thus quickly enters into the innermost detailed portions such as a hole of the item to be sterilized. As a result, the reliability of sterilization increases.

The humidifying step is performed by supplying vapors in the sterilizing chamber 10 using the humidifying apparatus 26 provided at the sterilizing chamber 10. The vapors penetrate into the innermost detail portions of such as a hole of the item to be sterilized through the humidifying step, and the high concentration NO₂ gas is filled under this state. Suitable humidity and NO₂ concentration for sterilization are achieved in the detailed and innermost portions of the item to be sterilized, and the reliability of the sterilization is preferably increased as a result. The combination of the sufficient humidity and NO₂ concentration facilitates the generation of nitric acid on the surface of germs, and increases the sterilizing effect. In addition to this, the high concentration NO₂ gas is filled after the humidification in the present Embodiment. Thus, in accordance with the pressure increase occurring when the high concentration NO₂ gas is filled in the sterilizing chamber 10, the NO₂ enters into the detailed and innermost portions of the already humidified item to be sterilized and is quickly converted to nitric acid. As a result, the sterilization effect is effectively achieved. In the present Embodiment, the humidification is performed under the decreased pressure through the evacuation. It is preferable that the generation of the vapors is therefore obtained in the humidifying apparatus 26 at a relatively low temperature, and vapors quickly enter into the detailed portions of the item to be sterilized.

In the supplying step, by firstly driving the NO₂ gas system, the high concentration NO₂ gas stored in the chamber 1 is sucked by the negative pressure in the sterilizing chamber 10 whose pressure has been decreased by the vacuuming step. The high concentration NO₂ gas passes through the supply pipe 10a with the opened first open/closure device 10b to be supplied to the sterilizing chamber 10. At the time of completing the supplying step, the first open/closure device 10b is closed.

In the exhausting step, the chamber 1 and the exhausting system communicate via the bypass pipe 25 by opening the third open/closure device 24. By driving the exhaust apparatus 17 of the exhaust system (air pump), the NO₂ gas remaining in the chamber 1 is sucked and is exhausted by making the gas harmless with the exhaust system. At the same time, the chamber 1 can be vacuumed (exhausting step) with the suction force. Therefore, in the present Embodiment, the NO₂ gas remaining in the chamber 1 can be exhausted by making the gas to be harmless, and the exhausting step (vacuuming) of the chamber 1 can be performed by the sucking function of the exhaust apparatus 17 of the same system. As such, it is advantageous that the sterilizing apparatus organically cooperates to function as a whole.

In the air charging step, a new dried gas mixture is sucked by the negative pressure of the chamber 1 which has been vacuumed in the exhausting step.

In the circulating step, the microwave generating apparatus 2a of the plasma generator 2 and the pressure device 6 are stared. With that, the gas mixture circulates in the circulating path 4, and nitrogen and oxygen of the gas mixture are displaced to the low-temperature plasma state in the plasma generating portion 2c of the plasma generator 2. As a result, nitrogen oxides such as nitrogen monoxide and nitrogen dioxide, and ozone are generated to generate an NOx gas mixture. By further circulating the NOx gas mixture, the NO₂ concentration is gradually increased as mentioned above, and the high concentration NO₂ gas with the NO₂ concentration of from 5,000 to 100,000 ppm is generated.

The high concentration NO₂ gas generated in the circulating step is supplied to the sterilizing chamber 10 by again performing the supplying step. In this manner, in the present Embodiment, by repeating the exhausting step, air charging step, circulating step, and supplying step, the internal pressure of the sterilizing chamber 10 with a larger volume than that of the chamber 1 which has been decreased in the vacuuming step increases, and the NO₂ concentration also gradually increases. By filling the high concentration NO₂ gas with the NO₂ concentration from 5,000 to 100,000 ppm, the NO₂ concentration in the sterilizing chamber 10 is adjusted to be from 9 to 100 mg/L, more preferably from 20 to 80 mg/L, and 20 to 40 mg/L in the present Embodiment. In the case the NO₂ concentration is less than 9 mg/L, a sufficient sterilization effect required for any germs cannot be obtained. On the other hand, in the case the concentration is above 100 mg/L, significant difference in shortening the sterilization time is not expected above such concentration, and rather, a problem associated with the exhaust gas treatment becomes troublesome.

In the sterilizing step, the item to be sterilized loaded by the setting step is maintained for a certain period of time in the sterilizing chamber 10 filled with the NO₂ gas with the predetermined NO₂ concentration. Although, the duration for sterilization is different depending on the factors such as the NO₂ concentration in the sterilizing chamber 10 and types of items to be sterilized, the sterilization is preferably maintained from 10 to 480 minutes. In the case the duration is less than 10 minutes, a sufficient sterilization effect required for any germs may not be obtained. On the other hand, in the case the duration is over 480 minutes, there is no significant difference in sterilization effect over such duration, and the processing time is likely to be unnecessarily prolonged.

In the gas exhausting step, the second open/closure device 23 is opened and the exhaust apparatus 17 of the exhausting system is driven. With that, the used high concentration NO₂ gas (exhaust gas) is sucked from the sterilizing chamber 10, and the NO₂ is removed and made to be harmless by the gas treatment means 15. In the present Embodiment, the exhaust gas remaining in the sterilizing chamber 10 is sucked and exhausted in part, i.e. approximately 3 to 10 times, with the predetermined NO₂ gas contents in accordance with the processing capacity of the gas treatment means 15. With that, it is advantageous that the exhaust gas treatment means does not need to be excessively configured even for the exhaust gas with high concentration NO₂, and the exhaust gas can be reliably made to be harmless.

The exhaust apparatus 17 is driven while the first open/closure device is closed and the second open/closure device is open to exhaust a certain amount of high concentration NO₂ gas in the sterilizing chamber to the exhaust gas treatment means 15 such that the pressure of the sterilizing chamber 10 is made to be negative. Subsequently, by opening the first open/closure device and closing the second open/closure device, the high concentration NO₂ gas remaining in the chamber 1 is sucked to the sterilizing chamber 10 by the negative pressure. It is possible that, by repeating the procedure for multiple times, the exhaust gas remaining in the sterilizing chamber 10 and chamber 1 is made to be harmless for exhaustion. With that, it is advantageous that the NO₂ gas remaining in the path connecting the chamber 1 and sterilizing chamber 10 (including the first open/closure device 10b) is also exhausted, and exhaustion of the entire sterilizing apparatus can be performed by effectively using the suction energy of the exhaust apparatus 17.

Hereinafter, the suitable mixture ratio between NO₂ and ozone in the exhaust system of the present invention was obtained by Examples.

### EXAMPLE

### Example 1

### Preparation of High Concentration NO₂ Gas

The high concentration NO₂ gas was prepared by the NO₂ gas supply system. The air (dew point: −60° C.) was used as an ingredient, and plasma lightning time in the plasma generator was 25 minutes. The concentration of the generated high concentration NO₂ was 47 kppm, and the gas was stored in the chamber. The pressure at this time, in terms of the differential pressure from the atmospheric pressure (101 kPa (absolute pressure)), was −5 kPa (relative pressure).

### (Preparation of Ozone)

By using an ozonizer (SGA-01-PSA2, manufactured by Sumitomo Precision Products Co., Ltd), ozone with 40 kppm was prepared using the air as an ingredient. The prepared ozone was introduced in the ozone chamber to perform substitution until the concentration of ozone reached 40 kppm.

### (Exhaust Step)

The exhaustion was started by opening the third open/closure device. After the high concentration NO₂ gas was passed through a particle filter (SFB200, manufactured by SMC Corporation) and a silica gel layer (silica gel A type 5UP, manufactured by Tokai Chemical Industry Co., Ltd.), the gas was adjusted by an exhaust gas flow meter (8550, manufactured by Kojima Instruments Inc.) such that the composition ratio relative to ozone was to be 2:1.

With an ozone flow controller (8500 manufactured by Kojima Instruments Inc.), the ozone was mixed with the above-described high concentration NO₂ gas, the flow of which had been adjusted, such that the composition ratio was to be 2:1. The mixing of the high concentration NO₂ gas and ozone was performed in the buffer portion (buffer tank). The gas mixture including dinitrogen pentoxide was passed through two nitric acid adsorption catalyst (ADS55, manufactured by Adsorption Technology Industries, Ltd.) disposed in serial. Subsequently, the gas mixture was sampled at times when the pressure in the chamber for storing the high concentration NO₂ showed certain values (−50, −65, −75, −85, and −90 kPa (relative pressure)) to measure the concentration of NOx remaining in the exhaust gas after passing the nitric acid adsorption catalyst. The results are shown in Table 1.

### Example 2

Other than that the concentration of the high concentration NO₂ gas generated in the NO₂ gas supply system was made to be 44 kppm, and the composition ratio of the high concentration NO₂ gas and ozone in the exhausting step was made to be 5:2, the high concentration NO₂ gas was processed in the same manner as Example 1 to measure the concentration of the remaining NOx. The results are shown in Table 1.

### Example 3

Other than that the concentration of the high concentration NO₂ gas generated in the NO₂ gas supply system was made to be 50 kppm, and the composition ratio of the high concentration NO₂ gas and ozone in the exhausting step was made to be 3:1, the high concentration NO₂ gas was processed in the same manner as Example 1 to measure the concentration of the remaining NOx. The results are shown in Table 1.

TABLE 1

| | | | | NOx | | NOx |
|---|---|---|---|---|---|---|
| | | % Ratio | % Ratio | concentration | | concentration |
| | NO₂:Ozone | of NO₂ | of NO₂ | before | Pressure in | after |
| | composition | (before | (after | treatment | chamber | treatment |
| | ratio | conversion) | conversion) | (ppm) | (kPa) | (ppm) |
| Example 1 | 2:1 | 67 | 60 | 47k | −50 | 0 |
| | | | | | −65 | 0 |
| | | | | | −75 | 0 |
| | | | | | −85 | 0 |
| | | | | | −90 | 0 |
| Example 2 | 5:2 | 71 | 65 | 44k | −50 | 0 |
| | | | | | −65 | 0 |
| | | | | | −75 | 0 |
| | | | | | −85 | 0 |
| | | | | | −90 | 0.14 |
| Example 3 | 3:1 | 75 | 69 | 50k | −50 | 0 |
| | | | | | −65 | 0 |
| | | | | | −75 | 0 |
| | | | | | −85 | 0 |
| | | | | | −90 | 1.33 |

As shown in Table 1, in Example 1 where the composition ratio of the high concentration NO₂ and ozone is 2:1, the NOx was completely absorbed in all cases when the internal pressures of the chamber were from −50 to −90 kPa (relative pressure). In Example 2 where the composition ratio of the high concentration NO₂ and ozone is 5:2, the NOx was also completely absorbed in all cases when the internal pressures of the chamber were from −50 to −90 kPa (relative pressure).

On the other hand, in Example 3 where the composition ratio of the high concentration NO₂ and ozone is 3:1, the NOx was completely absorbed in cases when the internal pressures of the chamber were from −50 to −85 kPa (relative pressure). However, a portion of NOx was not absorbed and remaining when the internal pressures of the chamber were −90 kPa (relative pressure).

The term, “% Ratio of NO₂ (before conversion)”, refers to a ratio of the high concentration NO₂ gas in the gas mixture of the high concentration NO₂ gas and ozone (theoretical value). In those Examples, a portion (25%) of the high concentration NO₂ gas is consumed by passing through the silica gel layer before being mixed with ozone. Thus, a ratio of the high concentration NO₂ gas in the gas mixture of the high concentration NO₂ gas and ozone (theoretical value) is calculated in “% Ratio of NO₂ (after conversion)”. Specifically, in terms of Example 1, the following calculations were performed.

% Ratio of NO₂ (before conversion): 2/(2+1)×100=67

% Ratio of NO₂ (after conversion): 2×0.75/(2×0.75+1)×100=60

From this, it is understood that NOx can be completely absorbed in the case the % ratio of NO₂ (after conversion) is smaller than 66% (Examples 1 and 2), and that a portion of NOx cannot be absorbed and remains in the case the % ratio of NO₂ (after conversion) is larger than 69% (Example 3).

### INDUSTRIAL APPLICABILITY

According to the exhaust system, the sterilizing apparatus using the exhaust system, and the sterilizing method using the sterilizing apparatus of the present invention, as a result of adsorbing NO₂ in a high concentration NO₂ gas used in a sterilization process and ozone, and generating nitric acid or dinitrogen pentoxide by accelerating the chemical reaction of the adsorbed NO₂ and ozone and retaining the resultant, the exhaust gas can be reliably and efficiently made to be harmless even if a concentration thereof is high.

According to the sterilizing apparatus of the present invention, NO₂ in the exhaust gas exhausted from the sterilizing apparatus can be removed. In addition, according to the sterilizing method of the present invention, high concentration NO₂ gas can be completely absorbed and exhausted after making the gas to be harmless.

### EXPLANATION OF SYMBOLS

1 chamber
2 plasma generator
2a microwave generating apparatus
2b waveguide
2c plasma generating portion
2d conducting shaft
2e conducting tube
2f antenna portion
2g center electrode
2h shielding film
2i ring-shaped space
2j pipe
2k shielding tube
3 circulating means
4 circulating path
5 flow resistive portion
5a orifice
6 pressure device
7 inlet portion
8 NO₂ concentration measurement sensor
9 gas drying means
10 sterilizing chamber
10a supply pipe
10b first open/closure device
11, 11a, 11b NO₂ sensor
12 first open/closure device
13 ozone generator
14 buffer portion
15 gas treatment means
16 ozone treatment means
17 exhaust apparatus
18 ozonizer
19 ozone chamber
20 ozone exhaust apparatus
21 ozone flow controller
22 exhaust pipe
23 second open/closure device
24 third open/closure device
25 bypass pipe
26 humidifying apparatus
D1 dehumidifying portion
F1 exhaust gas flow meter

1. An exhaust system for exhausting an exhaust gas used for sterilizing an item to be sterilized by using a high concentration NO₂ gas, comprising:
an ozone generator, a gas treatment means for adsorbing ozone generated by the ozone generator and NO₂ in the exhaust gas and accelerating generation of dinitrogen pentoxide or nitric acid by a reaction of the ozone and NO₂ to retain the resultant, and an exhaust apparatus for exhausting the exhaust gas.

2. The exhaust system according to claim 1, wherein the ozone generator comprises an ozonizer and an ozone chamber for storing ozone generated by the ozonizer.

3. The exhaust system according to claim 1, wherein a buffer portion for adjusting a mixture ratio of NO₂ in the exhaust gas and ozone is further provided at an upstream side from the gas treatment means.

4. The exhaust system according to claim 1, wherein the gas treatment means uses an adsorption catalyst.

5. The exhaust system according to claim 1, wherein the exhaust apparatus exhausts the exhaust gas used for sterilization in multiple times.

6. A sterilizing apparatus comprising:
(a) an NO₂ gas supply system configured by a circulating path in which a chamber for storing a high concentration NO₂ gas, a plasma generator, and a circulating means are connected;
(b) a sterilizing chamber connected to the chamber via a first open/closure device; and
(c) the exhaust system according to claim 1 connected to the sterilizing chamber via a second open/closure device.

7. The sterilizing apparatus according to claim 6, wherein the chamber is connected to the exhaust system via a third open/closure device.

8. The sterilizing apparatus according to claim 6, wherein the sterilizing chamber is provided with a measuring path for returning the high concentration NO₂ gas in the sterilizing chamber to the sterilizing chamber through an NO₂ sensor.

9. The sterilizing apparatus according to claim 6, wherein a plurality of sterilizing chambers is connected to a single exhaust system.

10. A sterilizing method using the sterilizing apparatus according to claim 6, wherein in a gas exhausting step for exhausting a high concentration NO₂ gas used for sterilizing an item to be sterilized, the high concentration NO₂ gas is exhausted to the exhaust system in part with a predetermined NO₂ gas contents.

11. A sterilizing method using the sterilizing apparatus according to claim 6, wherein in a gas exhausting step for exhausting a used high concentration NO₂ gas after a sterilizing step for sterilizing an item to be sterilized, a procedure of exhausting the high concentration NO₂ gas in the sterilizing chamber to the exhaust gas treatment means by an exhaust apparatus of the exhaust system to obtain a negative pressure in the sterilizing chamber while the first open/closure device is closed and the second open/closure device is open, and subsequently, sucking the high concentration NO₂ gas remaining the chamber into the sterilizing chamber by the negative pressure by opening the first open/closure device and closing the second open/closure device is repeated.

12. A sterilizing method using the sterilizing apparatus according to claim 6, comprising the steps of:
(d) setting an item to be sterilized in the sterilizing chamber (setting step);
(e) vacuuming the inside of the sterilizing chamber (vacuuming step);
(f) humidifying the inside of the sterilizing chamber (humidifying step);
(g) opening the first open/closure device to supply the NO₂ gas generated by the NO₂ gas system and stored in the chamber to the sterilizing chamber (supplying step);
(h) filling the dried gas mixture in the chamber (air charging step); and
(i) generating NO₂ gas by driving the NO₂ gas supply system (circulating step),
wherein the steps (g) to (i) are repeated a plurality of times.

13. The sterilizing method according to claim 12, wherein before the air charging step of step (h), a step of closing the first open/closure device and opening the third open/closure device to directly couple the chamber and exhaust system to exhaust the NO₂ gas remaining in the chamber with the exhaust system and to vacuum the chamber (gas exhausting step) is performed.
